(19) United States
(12) Patent Application Publication
(10) Pub. No.: US 2014/0200520 A1
(43) Pub. Date: Jul. 17, 2014

(54) METAL NEEDLE GUIDE

(71) Applicant: Marc Holtwick, Frankfurt am Main (DE)

(72) Inventor: Marc Holtwick, Frankfurt am Main (DE)

(73) Assignee: SANOFI-AVENTIS DEUTSCHLAND GMBH, Frankfurt am Main (DE)

(21) Appl. No.: 14/114,768

(22) PCT Filed: May 4, 2012
(86) PCT No.: PCT/EP2012/058269

§ 371 Date: Oct. 30, 2013

(30) Foreign Application Priority Data
May 6, 2011 (EP) 11165131.1

(51) Int. Cl. A61M 5/34; B21D 22/26

(52) U.S. Cl. CPC A61M 5/344; B21D 22/26
USPC 604/240; 72/347; 29/428

(57) ABSTRACT

The invention faces the technical problem of reducing the manufacturing costs of medical devices while at the same time a secure, quick and easy reception of a needle by said medical device is guaranteed. The technical problem is solved by a needle guide configured to receive a needle in a first opening in an axial direction of the needle guide, comprising a guide area comprising the first opening and a second opening, a flange area and a connection area, wherein said connection area is configured to non-detachably connect said needle guide to a medical device and wherein said needle guide is made of metal. The technical problem is further solved by a method to produce a needle guide, wherein said needle guide comprises a guide area, a flange area and a connection area, comprising the steps of deep drawing a metal sheet to produce the guide area.

### CROSS REFERENCE TO RELATED APPLICATIONS

The present application is a U.S. National Phase Application pursuant to 35 U.S.C. §371 of International Application No. PCT/EP2012/058269 filed May 4, 2012, which claims priority to European Patent Application No. 11165131.1 filed May 6, 2011. The entire disclosure contents of these applications are herewith incorporated by reference into the present application.

### FIELD OF DISCLOSURE

The present patent application relates to medical devices for delivering a drug from a reservoir. It also relates to a device for delivering at least two drug agents from separate reservoirs. Such drug agents may comprise a first and a second medicament. The medical device includes a dose setting mechanism for delivering the drug automatically or manually by the user.

The drug agents may be contained in one or more multiple dose reservoirs, containers or packages, each containing independent (single drug compound) or pre-mixed (co-formulated multiple drug compounds) drug agents.

### BACKGROUND

Certain disease states require treatment using one or more different medicaments. Some drug compounds need to be delivered in a specific relationship with each other in order to deliver the optimum therapeutic dose. The present patent application is of particular benefit where combination therapy is desirable, but not possible in a single formulation for reasons such as, but not limited to, stability, compromised therapeutic performance and toxicology.

For example, in some cases it might be beneficial to treat a diabetic with a long acting insulin (also may be referred to as the first or primary medicament) along with a glucagon-like peptide-1 such as GLP-1 or GLP-1 analog (also may be referred to as the second drug or secondary medicament).

Accordingly, there exists a need to provide devices for the delivery of two or more medicaments in a single injection or delivery step that is simple for the user to perform without complicated physical manipulations of the drug delivery device. The proposed drug delivery device provides separate storage containers or cartridge retainers for two or more active drug agents. These active drug agents are then only combined and/or delivered to the patient during a single delivery procedure. These active agents may be administered together in a combined dose or alternatively, these active agents may be combined in a sequential manner, one after the other.

### SUMMARY

The drug delivery device also allows for the opportunity of varying the quantity of the medicaments. For example, one fluid quantity can be varied by changing the properties of the injection device (e.g., setting a user variable dose or changing the device's “fixed” dose). The second medicament quantity can be changed by manufacturing a variety of secondary drug containing packages with each variant containing a different volume and/or concentration of the second active agent.

The drug delivery device may have a single dispense interface. This interface may be configured for fluid communication with the primary reservoir and with a secondary reservoir of medicament containing at least one drug agent. The drug dispense interface can be a type of outlet that allows the two or more medicaments to exit the system and be delivered to the patient.

The combination of compounds as discrete units or as a mixed unit can be delivered to the body via a double-ended needle assembly. This would provide a combination drug injection system that, from a user's perspective, would be achieved in a manner that closely matches the currently available injection devices that use standard needle assemblies. One possible delivery procedure may involve the following steps:

1. Attach a dispense interface to a distal end of the electro-mechanical injection device. The dispense interface comprises a first and a second proximal needle. The first and second needles pierce a first reservoir containing a primary compound and a second reservoir containing a secondary compound, respectively.

2. Attach a dose dispenser, such as a double-ended needle assembly, to a distal end of the dispense interface. In this manner, a proximal end of the needle assembly is in fluidic communication with both the primary compound and secondary compound.

3. Dial up/set a desired dose of the primary compound from the injection device, for example, via a graphical user interface (GUI).

4. After the user sets the dose of the primary compound, the micro-processor controlled control unit may determine or compute a dose of the secondary compound and preferably may determine or compute this second dose based on a previously stored therapeutic dose profile. It is this computed combination of medicaments that will then be injected by the user. The therapeutic dose profile may be user selectable.

5. Optionally, after the second dose has been computed, the device may be placed in an armed condition. In such an optional armed condition, this may be achieved by pressing and/or holding an “OK” button on a control panel. This condition may provide for greater than a predefined period of time before the device can be used to dispense the combined dose.

6. Then, the user will insert or apply the distal end of the dose dispenser (e.g., a double ended needle assembly) into the desired injection site. The dose of the combination of the primary compound and the secondary compound (and potentially a third medicament) is administered by activating an injection user interface (e.g., an injection button).

Both medicaments may be delivered via one injection needle or dose dispenser and in one injection step. This offers a convenient benefit to the user in terms of reduced user steps compared to administering two separate injections.

Especially for the production of aforementioned medical devices, for example the delivery device, the dose dispenser and in particular the dispense interface, it is important to provide devices which can be produced economically in mass production.

The dispense interface for example should not be used on different delivery devices and might need to be exchanged regularly even for a single delivery device in order to meet the hygienic standards. This makes such medical devices a mass product, where a simple and effective production needs to be achieved.

For hygienic reasons as well, a dose dispenser, such as a double-ended needle assembly, should be exchanged for every ejection process into the desired injection site, yielding in different exchanging frequencies for a dose dispenser and a dispense interface, for example. These different exchanging frequencies and the desire for flexibly attaching different dose dispensers, needles of different sizes for example, generate the need to produce corresponding modules, for example cartridge holder, dispense interface and dose dispenser, as separate attachable modules.

Especially the production of medical devices which need to receive a needle is comparatively expensive, since openings which are able to receive the needle securely need to be designed yielding in more complex structures for the receiving opening compared to the rest of the device.

The invention faces the technical problem of reducing the manufacturing costs of medical devices while at the same time a secure, quick and easy reception of a needle by said medical device is guaranteed.

The technical problem is solved by a needle guide configured to receive a needle in a first opening in an axial direction of the needle guide, comprising a guide area comprising the first opening and a second opening, a flange area and a connection area, wherein said connection area is configured to non-detachably connect said needle guide to a medical device and wherein said needle guide is made of metal.

By providing a needle guide the production of the needle guide can be separated from the production of the medical device to which the needle guide can be attached. This separation facilitates a very economic production of each element, the medical device and the needle guide. Here, needle is understood to mean any kind of needle or cannula.

The medical device can preferably be produced as a plastic part, for example by injection moulding. The production of said medical device can now take place without the need of producing the medical device and the needle guide directly as an integral part. By doing so, the production of the medical device is made simpler and more economical, since the comparatively complex geometry of a needle guide is especially disadvantageous for production methods like injection moulding.

By providing a guide area, the needle guide allows for a secure and easy guiding of the needle. The needle can preferably be inserted into the first opening or receiving opening of the guide area of the needle guide in the axial direction and the needle is then guided at least towards and preferably through the second opening of the guide area of the needle guide. The design of the guide area of the needle guide can precisely dictated the position of the needle, while the needle is being moved through the needle guide. This makes the handling easy and allows for a quick insertion of the needle into the needle guide. A precise positioning of the needle is important, since the dead volume of medical devices is often kept as small as possible resulting in small fluidic channels into which the needle needs to be inserted. The precise positioning can this way prevent the needle from not hitting the channel, which would result in not being able to establish a fluidic connection at all. Moreover the precise positioning can prevent the needle from colliding with the fluidic channels and causing leaks due to damaged channels.

The flange area is particularly advantageous for the connection of the needle guide to the medical device. By providing a flange area, preferably adjacent to the first opening of the guide area, the connection of the needle guide to the medical device is facilitated, because a force can be applied to the flange area of the needle guide and the needle guide can thus be pressed against the medical device to efficiently produce a connection without affecting the shape of the guide area, for example. It is also possible to non-detachably connect the needle guide to the medical device during the production process of the medical device. For example, during injection moulding the connection area of the medical device can be brought into the injection mould before the injection process.

The connection between a connection area of the needle guide and the medical device can generally be realised by form-fit, force-fit or by bonding. It is, for example, possible to provide pins, snap locks, threads or any type of glue to connect the needle guide to the medical device.

The connection area is preferably designed as a separate area and is preferably adjacent to the flange area and preferably provides connection means such as threads, hooks, or pins. But the connection area can also be the flange area itself, which is glued to the medical device, for example.

Since the needle guide itself does generally not need to be exchanged separately from the medical device it is attached non-detachably to the medical device. Accordingly means for a non-detachable connection are used.

The needle guide is furthermore made of metal, such as stainless steel, cold rolled steel or aluminium alloys, for example. This is especially advantageous, since needles can be guided effectively by metal surfaces due to the hardness of the material. Since needles are generally made of metal as well and comprising sharp edges, they cannot accidentally get stuck in the surface of a needle guide made of metal as it can happen in plastic needle guides. This further improves the handling of the medical device.

Moreover a needle guide made of metal can be effectively and economically produced by mass production, for example by forming, moulding or deep drawing.

It is especially advantageous, that the needle guide is made of metal, if the medical device at least in the area of the connection is made of plastic. This way the needle guide can effectively be fixed permanently. The connection elements can be in the form of a pin or of a barbed hook, which can be easily inserted into the plastic of the medical device by simply pushing it against the medical device.

The medical device can generally be any device, to which a needle should be attached or into which a needle should be inserted, in particular a dispense interface, a cartridge hub, a needle hub, or any combination of them.

It is a further advantage of the needle guide that it allows for more room in the medical device. A needle guide made of metal can achieve a better stability and rigidity than plastic with less material. Especially in connection with a dispense interface made from plastic, the use of unnecessary plastic material, which is resulting from production methods like injection moulding, in the area of the needle hub can be reduced by producing a metal needle guide.

It is especially advantageous, if said guide area is produced by deep drawing a metal sheet. The process of deep drawing is effective to produce small metal parts like the needle guide economically in mass production. Producing the needle guide by deep drawing may also comprise cleaning steps beforehand or further treatments like metal finishing, cutting or forming.

It is further preferred, if said guide area is substantially cylindrical in the axial direction and tapers at least in parts from the first opening of guide area to the second opening of the guide area. Substantially cylindrical in the axial direction means that the walls of the needle guide area are substantially cylindrical and the axis of the imaginary cylinder coincides with the axis of the axial direction. Due to the tapering the guide area is merely substantially cylindrical. That means that the first opening is larger than the second opening. The first opening of the guide area is preferably proximal to the flange area, while the second opening is distal to the flange area. The tapering allows for a very easy handling during the insertion of a needle into the needle guide area of the needle guide. The needle is forced to be inserted into the medical device at a defined point due to the tapering without needing the user to pay much attention to the process of insertion.

The guide area preferably tapers conically, producing the shape of a cone with a cut cone end. The user only needs to insert the needle into the broader first opening and the needle is positioned automatically with further progress of the insertion in the smaller second opening.

It is also advantageous, if the design of the tapering of the guide area is in a parabolic shape. The inner walls are shaped convex, while the curvature towards the second opening gets less. This may result in a smooth insertion of a needle into the guide area of the needle guide. Further, it may help the user, while inserting the needle into the needle guide, to gradually move the needle from a tilted position into a substantially parallel position relative to the axial direction and into a substantially concentric position relative to a fluidic channel, for example.

According to another embodiment of the needle guide, the flange area is adjacent to the guide area, said flange area is substantially circular and said flange area is extending substantially perpendicular to the axial direction. By designing the flange area in that way, a connection of the needle guide to the medical device is especially simple, which saves production costs. The flange area serves as face via which a force can by applied to the needle guide in the axial direction and the needle guide can thus be pressed against the medical device to efficiently produce a connection without deforming the shape of the needle guide, especially the guide area.

If said connection area extends substantially into the axial direction, an easy way of providing connection elements, which can engage the medical device in order to provide a connection by form fit, force fit or material bonding, is provided. Especially in connection with a flange area extending substantially perpendicular to the axial direction, connection elements in the connection area can engage the medical device easily.

According to another embodiment of the needle guide, said connection area comprises a plurality of projections separated by recesses substantially projecting in the axial direction. Such projections can engage the medical device and provide a permanent connection between medical device and needle guide. In particularly if the medical device is made of plastic in the area of the connection, the projections can be easily inserted into the plastic of the medical device. The medical device does not need to be prepared and the projections engaging the plastic connect the medical device with the needle guide.

The projections can generally be provided by a deep drawing process, but they can also be crimped, for example after a deep drawing process. If the needle guide is produced from a metal sheet the projections can already be cut into the metal sheet before deep drawing the guide area. It is also possible to produce the projections by cutting the recesses into a crimped rim in the connection area, for example by laser cutting.

It is further preferred if the recesses are tapering in the axial direction. This provides projections which are similar to a barbed hook, so that the projections can be easily inserted into the medical device, but provide a form-fit connection with the medical device in the engaged state. No further means for fixation need to be used, providing a very cost saving way of production. With an equidistant distribution of projections and recesses a uniform permanent connection can be provided.

According to a further embodiment of the needle guide, said projections are angled compared to the axial direction. This further increases the strength of the connection between needle guide and medical device. This way the angled projections act like dowels. The angle can increase during the engagement of the angled projections with the medical device and prevent the needle guide from being removed form or falling off the medical device.

It is in particular advantageous, if said projections are angled compared to the axial direction alternating towards the guide area and away from the guide area. This further increases the strength and thus the security of the connection between medical device and needle guide.

The technical problem is also solved by an apparatus comprising a needle guide according to the invention, wherein said apparatus is a medical device configured to eject at least one medicament, or a dispense interface attachable to a medical device configured to eject at least one medicament.

The technical problem is further solved by a method to produce a needle guide, wherein said needle guide comprises a guide area, a flange area and a connection area, comprising the steps of deep drawing a metal sheet to produce the guide area. By producing the needle guide by deep drawing a metal sheet, the production of the needle guide is decoupled from the production of medical devices for which the needle guide can be used for. Especially deep drawing provides an economic way for mass production processes and is cost saving compared to producing medical devices integral with the needle guide. By using a metal sheet for deep drawing a needle guide is provided which has an improved rigidity and stability compared to plastic needle guides.

It is further advantageous, if said guide area, said flange area and said connection area are produced at least in part by said step of deep drawing said metal sheet. By producing the guide area, the flange area and the connection area by a deep drawing step the production of the needle guide is further facilitated due to a minimum of process steps. Though, further processing of the needle guide is possible. These processing steps can include crimping, mechanical cutting, laser cutting, and metal treatments before and/or after the deep drawing. Preferably said flange area and said connection area are produced completely by said step of deep drawing said metal sheet.

According to another embodiment of the method, said method further comprises the step of pressing said needle guide into a medical device. By pressing the needle guide into the medical device a non-detachable connection between the needle guide and the medical device is established, without the need of glue or threads. The connection is achieved by form-fit and/or force-fit between the connection area of the needle guide and the medical device. It is especially advantageous, if the medical device, at least in the area of connection, is made from plastic. This facilitates the engagement of the connection area into the medical device. Connection elements according to the needle guide according to the invention can be used.

According to another embodiment of the method, said medical device is a dispense interface, in particular a needle hub. It is particularly advantageous to attach a needle guide according to the invention to a dispense interface. Dispense interfaces are used, for example, to provide a single outlet for a device comprising two fluids, in particular medicaments. A dose dispenser comprises a needle, for example a double ended needle, which can be connected to the dispense interface. The dose dispenser needs to be exchanged regularly by the user, which makes it particularly advantageous to provide a needle guide, which provides an easy and reliable attachment of the dose dispenser to the dispense interface for the user, while at the same a cost saving method for the production of the needle guide and thus of the dispense interface is provided.

For further designs and advantages of the method according to the invention, it is referred to the description of the needle guide according to the invention.

### BRIEF DESCRIPTION OF THE FIGURES

These as well as other advantages of various aspects of the present invention will become apparent to those of ordinary skill in the art by reading the following detailed description, with appropriate reference to the accompanying drawings, in which:

FIG. 1 illustrates a perspective view of the delivery device illustrated in Fig. la and lb with an end cap of the device removed;

FIG. 2 illustrates a perspective view of the delivery device distal end showing the cartridge;

FIG. 3 illustrates a perspective view of the cartridge holder illustrated in FIG. 1 with one cartridge retainer in an open position;

FIG. 4 illustrates a dispense interface and a dose dispenser that may be removably mounted on a distal end of the delivery device illustrated in FIG. 1;

FIG. 5 illustrates the dispense interface and the dose dispenser illustrated in FIG. 4 mounted on a distal end of the delivery device illustrated in FIG. 1;

FIG. 6 illustrates one arrangement of the dose dispenser that may be mounted on a distal end of the delivery device;

FIG. 7 illustrates a perspective view of the dispense interface illustrated in FIG. 4;

FIG. 8 illustrates another perspective view of the dispense interface illustrated in FIG. 4;

FIG. 9 illustrates a cross-sectional view of the dispense interface illustrated in FIG. 4;

FIG. 10 illustrates an exploded view of the dispense interface illustrated in FIG. 4;

FIG. 11 illustrates a cross-sectional perspective view of the dispense interface and dose dispenser mounted onto a drug delivery device, such as the device illustrated in FIG. 1;

FIG. 12 illustrates a cross sectional view of an embodiment of a needle guide according to the invention;

FIG. 13 illustrates different embodiments of connection areas of an exemplary needle guide;

FIG. 14 illustrates a cross sectional view of an embodiment of the needle guide attached to an exemplary needle hub of a dispense interface and an exemplary dose dispenser.

FIG. 15 illustrates a cross sectional view of a further embodiment of a needle guide attached to an exemplary needle hub of a dispense interface.

### DETAILED DESCRIPTION

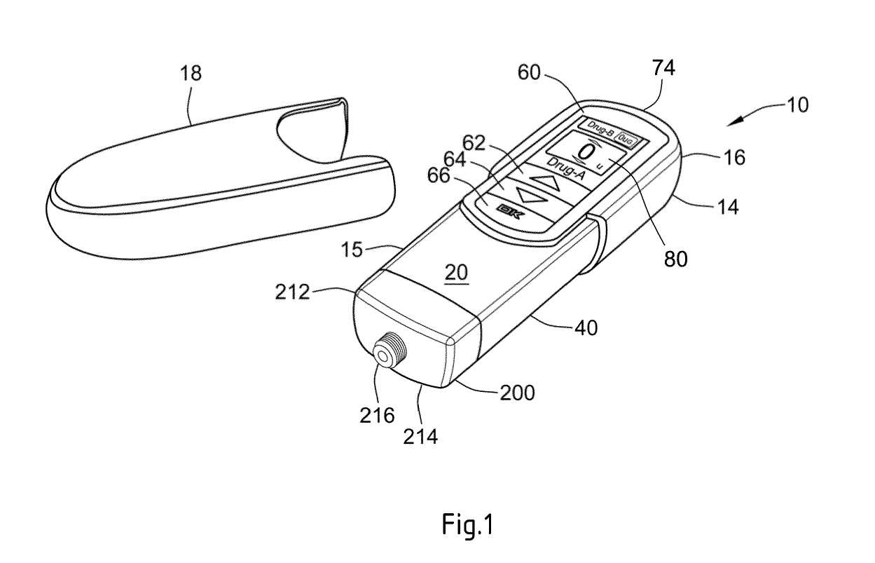

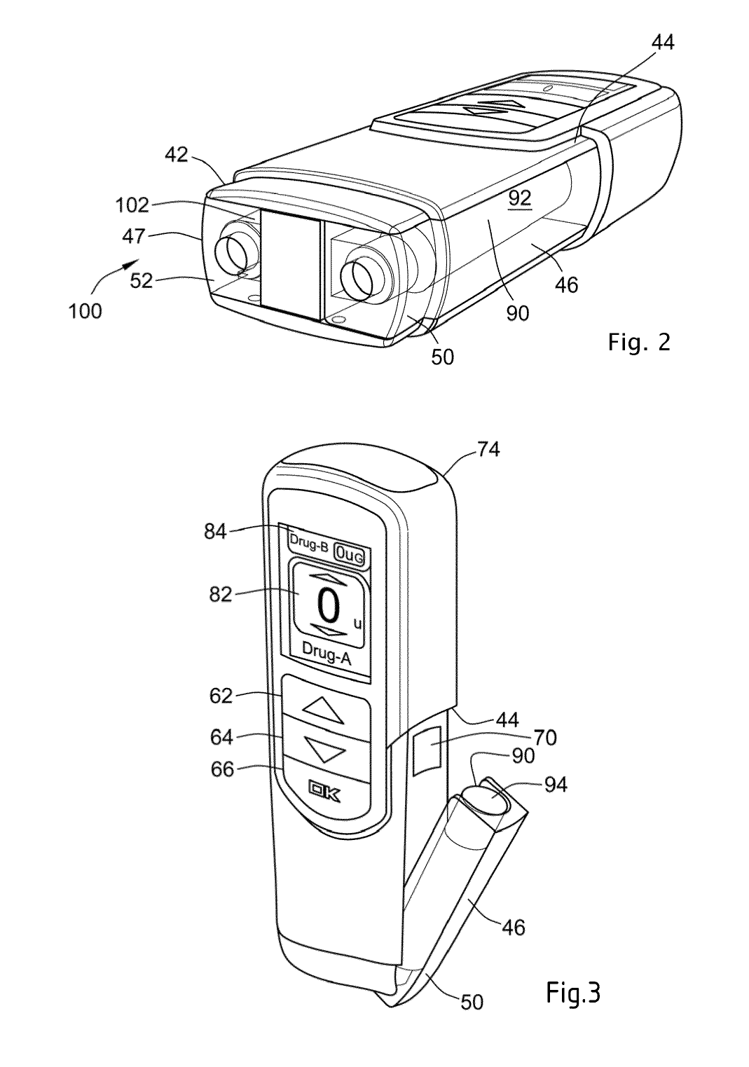

The drug delivery device illustrated in FIG. 1 comprises a main body 14 that extends from a proximal end 16 to a distal end 15. At the distal end 15, a removable end cap or cover 18 is provided. This end cap 18 and the distal end 15 of the main body 14 work together to provide a snap fit or form fit connection so that once the cover 18 is slid onto the distal end 15 of the main body 14, this frictional fit between the cap and the main body outer surface 20 prevents the cover from inadvertently falling off the main body.

The main body 14 contains a micro-processor control unit, an electro-mechanical drive train, and at least two medicament reservoirs. When the end cap or cover 18 is removed from the device 10 (as illustrated in FIG. 1), a dispense interface 200 is mounted to the distal end 15 of the main body 14, and a dose dispenser (e.g., a needle assembly) is attached to the interface. The drug delivery device 10 can be used to administer a computed dose of a second medicament (secondary drug compound) and a variable dose of a first medicament (primary drug compound) through a single needle assembly, such as a double ended needle assembly.

A control panel region 60 is provided near the proximal end of the main body 14. Preferably, this control panel region 60 comprises a digital display 80 along with a plurality of human interface elements that can be manipulated by a user to set and inject a combined dose. In this arrangement, the control panel region comprises a first dose setting button 62, a second dose setting button 64 and a third button 66 designated with the symbol “OK.” In addition, along the most proximal end of the main body, an injection button 74 is also provided (not visible in the perspective view of FIG. 1).

The cartridge holder 40 can be removably attached to the main body 14 and may contain at least two cartridge retainers 50 and 52. Each retainer is configured so as to contain one medicament reservoir, such as a glass cartridge. Preferably, each cartridge contains a different medicament.

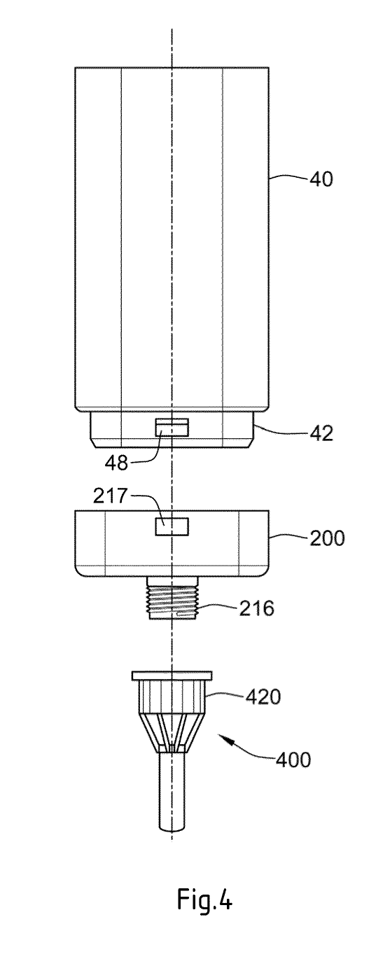

In addition, at the distal end of the cartridge holder 40, the drug delivery device illustrated in FIG. 1 includes a dispense interface 200. As will be described in relation to FIG. 4, in one arrangement, this dispense interface 200 includes a main outer body 212 that is removably attached to a distal end 42 of the cartridge housing 40. As can be seen in FIG. 1, a distal end 214 of the dispense interface 200 preferably comprises a needle hub 216. This needle hub 216 may be configured so as to allow a dose dispenser, such as a conventional pen type injection needle assembly, to be removably mounted to the drug delivery device 10.

Once the device is turned on, the digital display 80 shown in FIG. 1 illuminates and provides the user certain device information, preferably information relating to the medicaments contained within the cartridge holder 40. For example, the user is provided with certain information relating to both the primary medicament (Drug A) and the secondary medicament (Drug B).

As shown in FIG. 3, the first and a second cartridge retainers 50, 52 comprise hinged cartridge retainers. These hinged retainers allow user access to the cartridges. FIG. 3 illustrates a perspective view of the cartridge holder 40 illustrated in FIG. 1 with the first hinged cartridge retainer 50 in an open position. FIG. 3 illustrates how a user might access the first cartridge 90 by opening up the first retainer 50 and thereby having access to the first cartridge 90.

As mentioned above when discussing FIG. 1, a dispense interface 200 is coupled to the distal end of the cartridge holder 40. FIG. 4 illustrates a flat view of the dispense interface 200 unconnected to the distal end of the cartridge holder 40. A dose dispenser or needle assembly that may be used with the interface 200 is also illustrated and is provided in a protective outer cap 420.

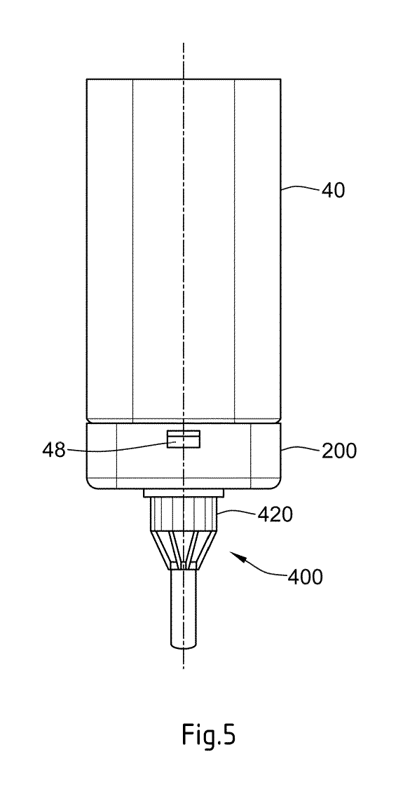

In FIG. 5, the dispense interface 200 illustrated in FIG. 4 is shown coupled to the cartridge holder 40. The axial attachment means between the dispense interface 200 and the cartridge holder 40 can be any known axial attachment means to those skilled in the art, including snap locks, snap fits, snap rings, keyed slots, and combinations of such connections. The connection or attachment between the dispense interface and the cartridge holder may also contain additional features (not shown), such as connectors, stops, splines, ribs, grooves, pips, clips and the like design features, that ensure that specific hubs are attachable only to matching drug delivery devices. Such additional features would prevent the insertion of a non-appropriate secondary cartridge to a non-matching injection device.

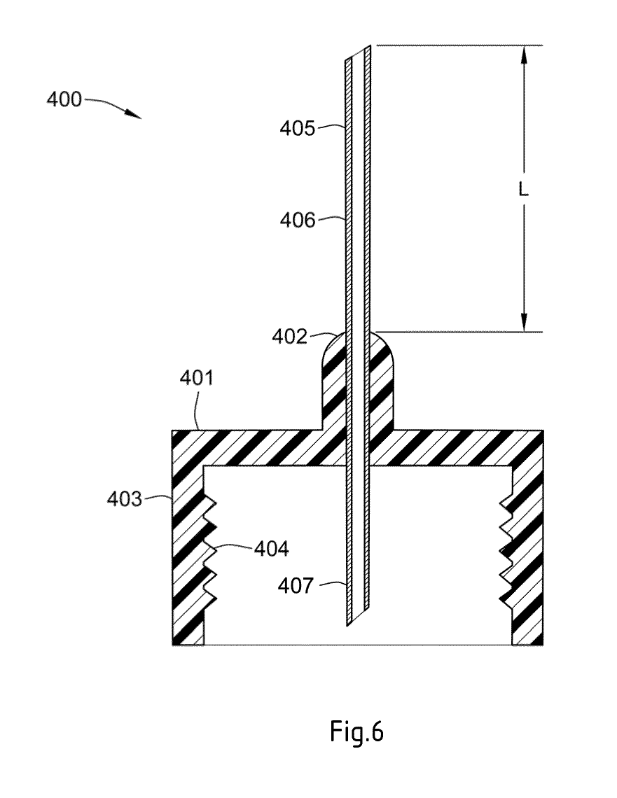

FIG. 5 also illustrates the needle assembly 400 and protective cover 420 coupled to the distal end of the dispense interface 200 that may be screwed onto the needle hub of the interface 200. FIG. 6 illustrates a cross sectional view of the double ended needle assembly 402 mounted on the dispense interface 200 in FIG. 5.

The needle assembly 400 illustrated in FIG. 6 comprises a double ended needle 406 and a hub 401. The double ended needle or cannula 406 is fixedly mounted in a needle hub 401. This needle hub 401 comprises a circular disk shaped element which has along its periphery a circumferential depending sleeve 403. Along an inner wall of this hub member 401, a thread 404 is provided. This thread 404 allows the needle hub 401 to be screwed onto the dispense interface 200 which, in one preferred arrangement, is provided with a corresponding outer thread along a distal hub. At a center portion of the hub element 401 there is provided a protrusion 402. This protrusion 402 projects from the hub in an opposite direction of the sleeve member. A double ended needle 406 is mounted centrally through the protrusion 402 and the needle hub 401. This double ended needle 406 is mounted such that a first or distal piercing end 405 of the double ended needle forms an injecting part for piercing an injection site (e.g., the skin of a user).

Similarly, a second or proximal piercing end 406 of the needle assembly 400 protrudes from an opposite side of the circular disc so that it is concentrically surrounded by the sleeve 403. In one needle assembly arrangement, the second or proximal piercing end 406 may be shorter than the sleeve 403 so that this sleeve to some extent protects the pointed end of the back sleeve. The needle cover cap 420 illustrated in FIGS. 4 and 5 provides a form fit around the outer surface 403 of the hub 401.

Referring now to FIGS. 4 to 11, one preferred arrangement of this interface 200 will now be discussed. In this one preferred arrangement, this interface 200 comprises:
a. a main outer body 210,
b. an first inner body 220,
c. a second inner body 230,
d. a first piercing needle 240,
e. a second piercing needle 250,
f. a valve seal 260, and
g. a septum 270.

The main outer body 210 comprises a main body proximal end 212 and a main body distal end 214. At the proximal end 212 of the outer body 210, a connecting member is configured so as to allow the dispense interface 200 to be attached to the distal end of the cartridge holder 40. Preferably, the connecting member is configured so as to allow the dispense interface 200 to be removably connected the cartridge holder 40. In one preferred interface arrangement, the proximal end of the interface 200 is configured with an upwardly extending wall 218 having at least one recess. For example, as may be seen from FIG. 8, the upwardly extending wall 218 comprises at least a first recess 217 and a second recess 219.

Preferably, the first and the second recesses 217, 219 are positioned within this main outer body wall so as to cooperate with an outwardly protruding member located near the distal end of the cartridge housing 40 of the drug delivery device 10. For example, this outwardly protruding member 48 of the cartridge housing may be seen in FIGS. 4 and 5. A second similar protruding member is provided on the opposite side of the cartridge housing. As such, when the interface 200 is axially slid over the distal end of the cartridge housing 40, the outwardly protruding members will cooperate with the first and second recess 217, 219 to form an interference fit, form fit, or snap lock. Alternatively, and as those of skill in the art will recognize, any other similar connection mechanism that allows for the dispense interface and the cartridge housing 40 to be axially coupled could be used as well.

The main outer body 210 and the distal end of the cartridge holder 40 act to form an axially engaging snap lock or snap fit arrangement that could be axially slid onto the distal end of the cartridge housing. In one alternative arrangement, the dispense interface 200 may be provided with a coding feature so as to prevent inadvertent dispense interface cross use. That is, the inner body of the hub could be geometrically configured so as to prevent an inadvertent cross use of one or more dispense interfaces.

A mounting hub is provided at a distal end of the main outer body 210 of the dispense interface 200. Such a mounting hub can be configured to be releasably connected to a needle assembly. As just one example, this connecting means 216 may comprise an outer thread that engages an inner thread provided along an inner wall surface of a needle hub of a needle assembly, such as the needle assembly 400 illustrated in FIG. 6. Alternative releasable connectors may also be provided such as a snap lock, a snap lock released through threads, a bayonet lock, a form fit, or other similar connection arrangements.

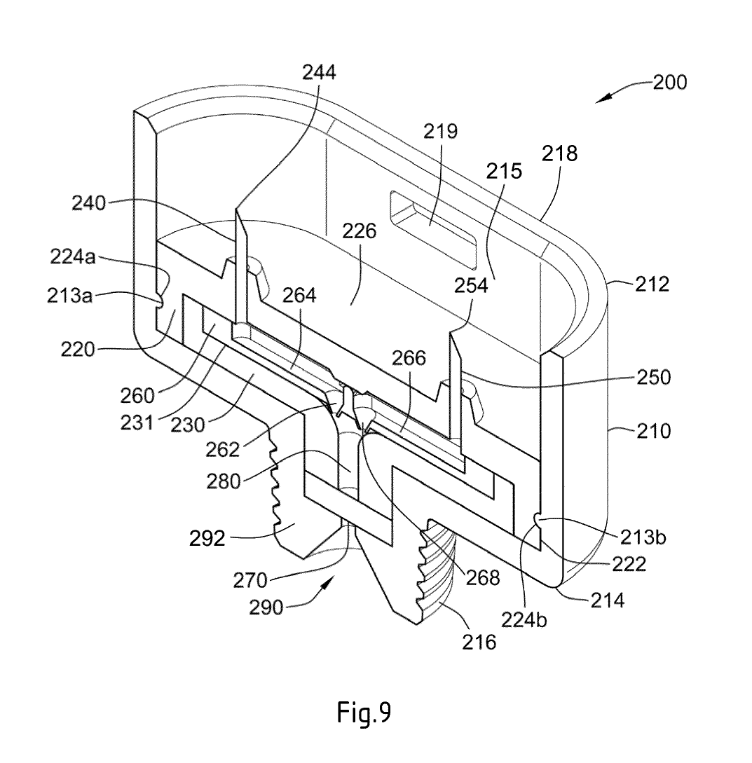

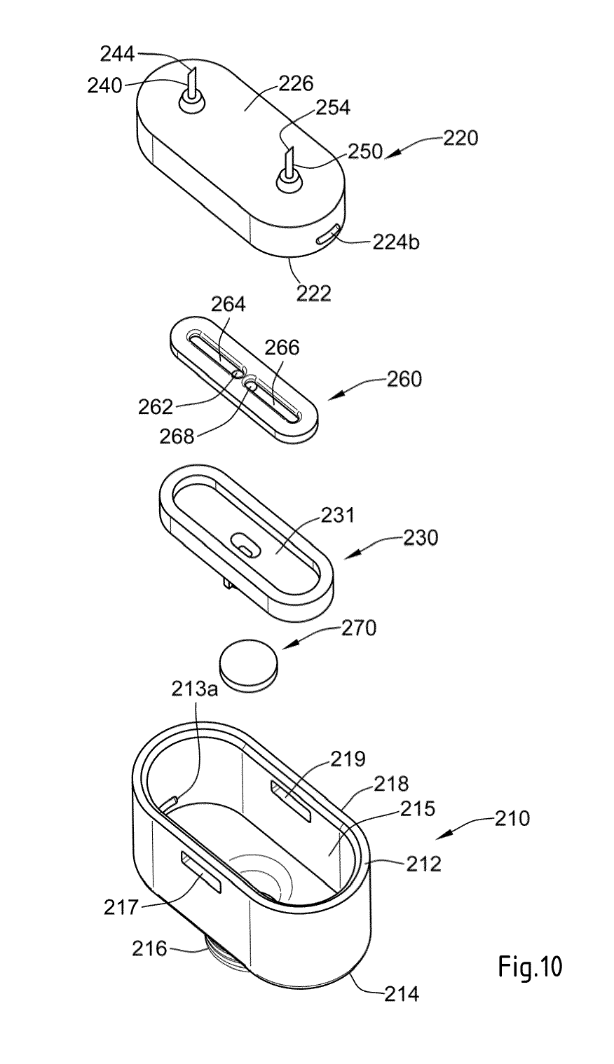

The dispense interface 200 further comprises a first inner body 220. Certain details of this inner body are illustrated in FIG. 8-11. Preferably, this first inner body 220 is coupled to an inner surface 215 of the extending wall 218 of the main outer body 210. More preferably, this first inner body 220 is coupled by way of a rib and groove form fit arrangement to an inner surface of the outer body 210. For example, as can be seen from FIG. 9, the extending wall 218 of the main outer body 210 is provided with a first rib 213a and a second rib 213b. This first rib 213a is also illustrated in FIG. 10. These ribs 213a and 213b are positioned along the inner surface 215 of the wall 218 of the outer body 210 and create a form fit or snap lock engagement with cooperating grooves 224a and 224b of the first inner body 220. In a preferred arrangement, these cooperating grooves 224a and 224b are provided along an outer surface 222 of the first inner body 220.

In addition, as can be seen in FIG. 8-10, a proximal surface 226 near the proximal end of the first inner body 220 may be configured with at least a first proximally positioned piercing needle 240 comprising a proximal piercing end portion 244. Similarly, the first inner body 220 is configured with a second proximally positioned piercing needle 250 comprising a proximally piercing end portion 254. Both the first and second needles 240, 250 are rigidly mounted on the proximal surface 226 of the first inner body 220.

Preferably, this dispense interface 200 further comprises a valve arrangement. Such a valve arrangement could be constructed so as to prevent cross contamination of the first and second medicaments contained in the first and second reservoirs, respectively. A preferred valve arrangement may also be configured so as to prevent back flow and cross contamination of the first and second medicaments.

In one preferred system, dispense interface 200 includes a valve arrangement in the form of a valve seal 260. Such a valve seal 260 may be provided within a cavity 231 defined by the second inner body 230, so as to form a holding chamber 280. Preferably, cavity 231 resides along an upper surface of the second inner body 230. This valve seal comprises an upper surface that defines both a first fluid groove 264 and second fluid groove 266. For example, FIG. 9 illustrates the position of the valve seal 260, seated between the first inner body 220 and the second inner body 230. During an injection step, this seal valve 260 helps to prevent the primary medicament in the first pathway from migrating to the secondary medicament in the second pathway, while also preventing the secondary medicament in the second pathway from migrating to the primary medicament in the first pathway. Preferably, this seal valve 260 comprises a first non-return valve 262 and a second non-return valve 268. As such, the first non-return valve 262 prevents fluid transferring along the first fluid pathway 264, for example a groove in the seal valve 260, from returning back into this pathway 264. Similarly, the second non-return valve 268 prevents fluid transferring along the second fluid pathway 266 from returning back into this pathway 266.

Together, the first and second grooves 264, 266 converge towards the non-return valves 262 and 268 respectively, to then provide for an output fluid path or a holding chamber 280. This holding chamber 280 is defined by an inner chamber defined by a distal end of the second inner body both the first and the second non return valves 262, 268 along with a pierceable septum 270. As illustrated, this pierceable septum 270 is positioned between a distal end portion of the second inner body 230 and an inner surface defined by the needle hub of the main outer body 210.

The holding chamber 280 terminates at an outlet port of the interface 200. This outlet port 290 is preferably centrally located in the needle hub of the interface 200 and assists in maintaining the pierceable seal 270 in a stationary position. It is observable though, that the needle guide 292 takes up a comparably large part of the room of the outlet port 290 and the needle hub 216. As such, when a double ended needle assembly is attached to the needle hub of the interface (such as the double ended needle illustrated in FIG. 6), the output fluid path allows both medicaments to be in fluid communication with the attached needle assembly.

The hub interface 200 further comprises a second inner body 230. As can be seen from FIG. 9, this second inner body 230 has an upper surface that defines a recess, and the valve seal 260 is positioned within this recess. Therefore, when the interface 200 is assembled as shown in FIG. 9, the second inner body 230 will be positioned between a distal end of the outer body 210 and the first inner body 220. Together, second inner body 230 and the main outer body hold the septum 270 in place. The distal end of the inner body 230 may also form a cavity or holding chamber that can be configured to be fluid communication with both the first groove 264 and the second groove 266 of the valve seal.

Axially sliding the main outer body 210 over the distal end of the drug delivery device attaches the dispense interface 200 to the multi-use device. In this manner, a fluid communication may be created between the first needle 240 and the second needle 250 with the primary medicament of the first cartridge and the secondary medicament of the second cartridge, respectively.

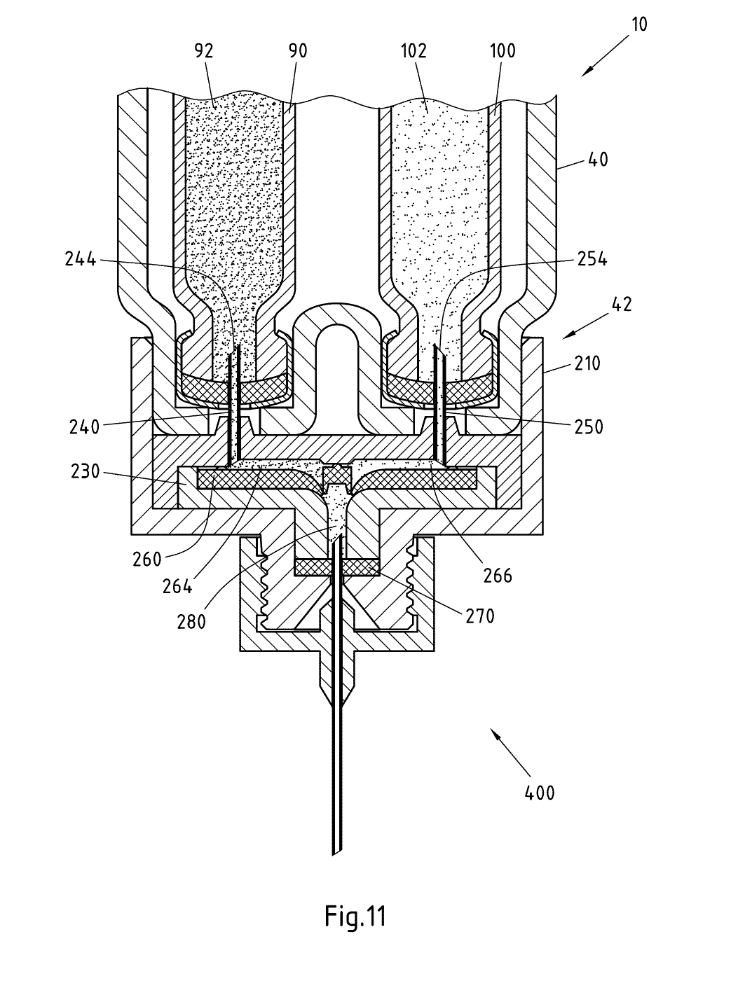

FIG. 11 illustrates the dispense interface 200 after it has been mounted onto the distal end 42 of the cartridge holder 40 of the drug delivery device 10 illustrated in FIG. 1. A double ended needle 400 is also mounted to the distal end of this interface. The cartridge holder 40 is illustrated as having a first cartridge containing a first medicament and a second cartridge containing a second medicament.

When the interface 200 is first mounted over the distal end of the cartridge holder 40, the proximal piercing end 244 of the first piercing needle 240 pierces the septum of the first cartridge 90 and thereby resides in fluid communication with the primary medicament 92 of the first cartridge 90. A distal end of the first piercing needle 240 will also be in fluid communication with a first fluid path groove 264 defined by the valve seal 260.

Similarly, the proximal piercing end 254 of the second piercing needle 250 pierces the septum of the second cartridge 100 and thereby resides in fluid communication with the secondary medicament 102 of the second cartridge 100. A distal end of this second piercing needle 250 will also be in fluid communication with a second fluid path groove 266 defined by the valve seal 260.

FIG. 11 illustrates a preferred arrangement of such a dispense interface 200 that is coupled to a distal end 15 of the main body 14 of drug delivery device 10. Preferably, such a dispense interface 200 is removably coupled to the cartridge holder 40 of the drug delivery device 10.

As illustrated in FIG. 11, the dispense interface 200 is coupled to the distal end of a cartridge housing 40. This cartridge holder 40 is illustrated as containing the first cartridge 90 containing the primary medicament 92 and the second cartridge 100 containing the secondary medicament 102. Once coupled to the cartridge housing 40, the dispense interface 200 essentially provides a mechanism for providing a fluid communication path from the first and second cartridges 90, 100 to the common holding chamber 280. This holding chamber 280 is illustrated as being in fluid communication with a dose dispenser. Here, as illustrated, this dose dispenser comprises the double ended needle assembly 400. As illustrated, the proximal end of the double ended needle assembly is in fluid communication with the chamber 280.

In one preferred arrangement, the dispense interface is configured so that it attaches to the main body in only one orientation, that is it is fitted only one way round. As such as illustrated in FIG. 11, once the dispense interface 200 is attached to the cartridge holder 40, the primary needle 240 can only be used for fluid communication with the primary medicament 92 of the first cartridge 90 and the interface 200 would be prevented from being reattached to the holder 40 so that the primary needle 240 could now be used for fluid communication with the secondary medicament 102 of the second cartridge 100. Such a one way around connecting mechanism may help to reduce potential cross contamination between the two medicaments 92 and 102.

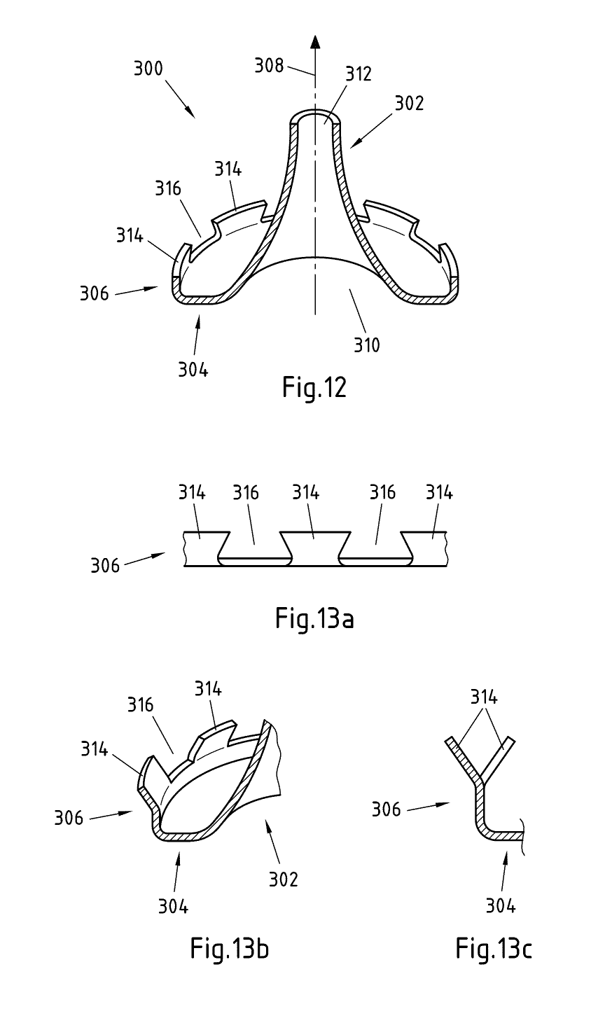

FIG. 12 illustrates a cross sectional perspective view of an embodiment of a needle guide 300 according to the invention. The needle guide 300 comprises a guide area 302, a flange area 304 and a connection area 306. As can be seen, the guide area extends in the axial direction, illustrated by the arrow 308. The guide area further comprises a first opening 310 and a second opening 312. The first opening 310 can receive a needle 406. The guide area 302 then guides the needle to the second opening 312 during a movement of the needle 406 in the axial direction 308. In case the needle 406 is not coaxially inserted into the needle guide 300, the geometric shape of the guide area 302 guides the needle 406 to the second opening 312, which has a similar diameter to the diameter of the needle 406, bringing the axis of the needle 406 substantially in coincidence with the arrow 308. The parabolic shape of the guide area 302 guides the needle 406 particularly smooth, other shapes, like conical, cylindrical, concave, convex or the like, are possible, too.

As can be further seen from FIG. 12, the flange area 304 extends substantially perpendicular to the axial direction 308. This way an area for pressing the needle guide 300 into a medical device is provided.

The connection area 306 further provides a plurality of projections 314 extending in the axial direction 308. The projections 314 are used as connection elements and can engage a medical device, like a dispense interface 200, made from plastic, for example. The projections 314 are forced into the medical device when enough pressure is exerted onto the flange area 304. The single projections 314 are separated by recesses 316. The recesses reduce the force necessary to press the projections 314 of the needle guide 300 in the medical device. The recesses 316 taper in the axial direction 308 providing the recesses 316 with the shape of a barbed hook or a dowel, which positively affects the durability of the connection between needle guide 300 and medical device. Other shapes of projections 314, like pins or blades, are possible as well. The needle guide 300 illustrated in FIG. 12 can be produced especially economically by deep drawing a metal sheet.

FIG. 13 illustrates different embodiments of connection areas 306 of an exemplary needle guide 300. FIG. 13a illustrates the enlarged connection area 306 illustrated in FIG. 12 in a side view. The tapering of the recesses 316 between the projections 314 can be seen clearly.

FIG. 13b illustrates another embodiment of a connection area 306 extending perpendicular from the flange area 304. Here, the projections 314 have a rectangular shape and the recesses 316 are not tapered. It can be seen that the projections 314 are angled compared to the axial direction 308 alternating towards the guide area 302 and away from the guide area 302. In FIG. 13c the alternating angled projections 314 can be seen in a cross sectional view of the connection area 306 from FIG. 13b. The alternating projections 314 are particularly effective in securing the needle guide 300 from getting removed from the medical device.

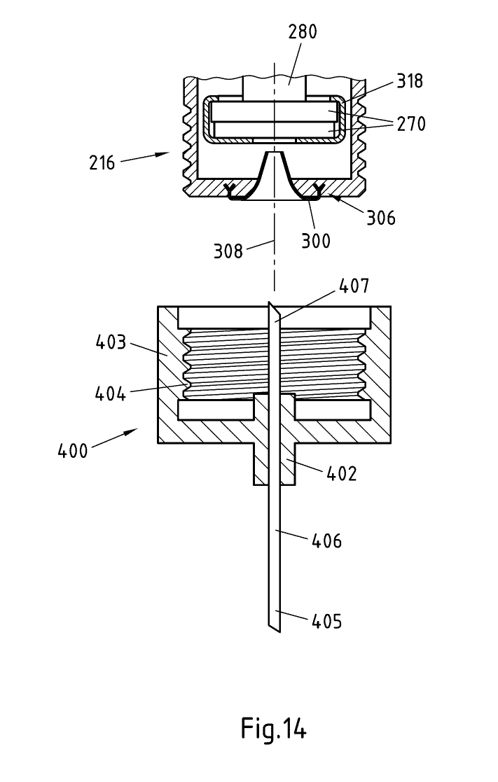

FIG. 14 illustrates a cross sectional view of an embodiment of the needle guide 300 attached to an exemplary needle hub 216 of a dispense interface 200 and an exemplary dose dispenser 400. The dose dispenser 400 is similar to the one illustrated in FIG. 6. The dose dispenser 400 comprises a double ended needle 406. The double ended needle or cannula 406 is fixedly mounted in the protrusion 402. The dose dispenser 400 comprises a circular disk shaped element which has along its periphery a circumferential depending sleeve 403. Along an inner wall of the dose dispenser 400, a thread 404 is provided. This thread 404 allows the dose dispenser 400 to be screwed onto the needle hub 216 of the dispense interface 200 which, in one preferred arrangement, is provided with a corresponding outer thread along the needle hub 216. The double ended needle 406 is mounted such that a first or distal piercing end 405 of the double ended needle forms an injecting part for piercing an injection site (e.g., the skin of a user).

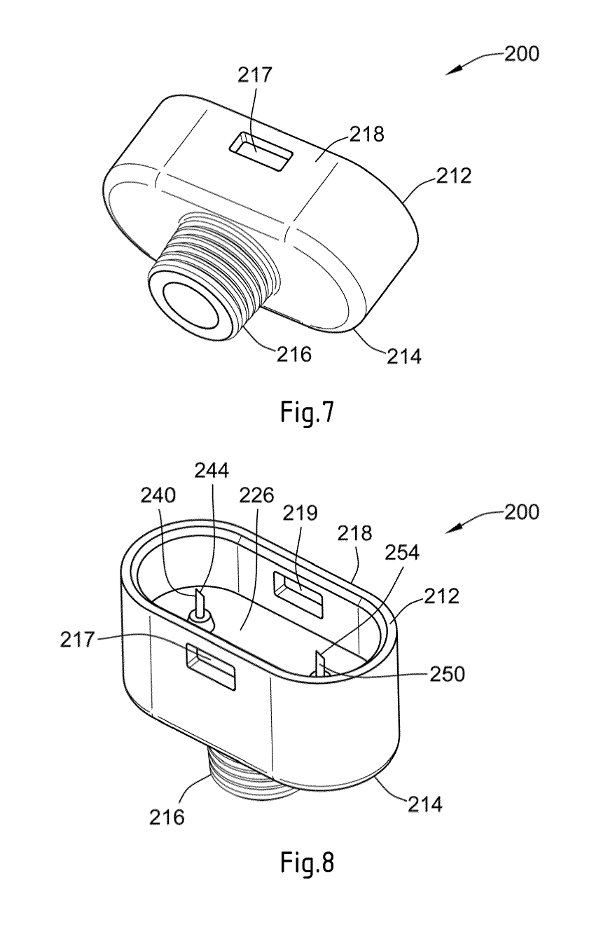

The second end 407 of the needle or cannula 406 is guided by the needle guide 300 into the needle hub 216 of a drug delivery device, for example the dose dispenser 200 illustrated in FIG. 7 or 8. Of course, the needle guide 300 can also be used with a drug delivery device or a dispense interface, which is configured to eject a single medicament or liquid. It can be seen that the connection area 306 of the needle guide is non-detachably connected to the needle hub 216. In case the needle 406 has an offset from the axis coinciding with the axial direction 308, the needle 406 is guided by the guide area 302 so that the axis of the needle 406 and the axis coinciding with the axial direction 308 coincide. The needle 406 can then accurately pierce one or more septa 270, which seal a holding chamber 280 in this case. The two septa are fixed by a clip 318.

Moreover, it can be seen that compared to the needle guide 292 illustrated in FIG. 9 the needle guide 300 takes up less space. This allows for further elements to be implemented in the dispense interface 200 or a lighter and smaller design of the dispense interface 200.

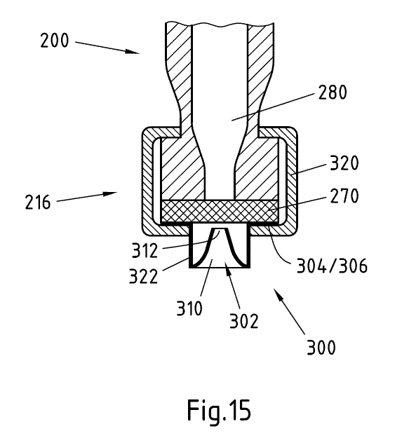

FIG. 15 illustrates a cross sectional view of a further embodiment of a needle guide 300 attached to an exemplary needle hub 216 of a dispense interface 200. The embodiment illustrated in FIG. 15 differs from the embodiment illustrated in FIG. 14 in that the septum 270 and the need to guide 300 are non-detachably attached to the dispense interface 200 in the area of the holding chamber 280 by the crimping clip 320. The crimping clip 320 encompasses a part of the dispense interface 200, the septum 270 and the needle guide 300. The dispense interface 200 provides in this case adapted recesses with which the crimping clip 320 can engage in order to be fixedly mounted on the dispense interface 200. The crimping of the crimping clip 320 can in principle not be reversed or only be reversed with great effort and/or with damaging certain components.

The needle guide 300 illustrated in FIG. 15 particularly differs from the needle guide 300 illustrated in FIG. 12 in that the flange area 304 is at the same time the connection area 306. The flange and connection area 304, 306 are located at the opposite end of the guide area 302 at the second opening 312. Flange and connection area 304, 306 are connected to the guide area 302 by a cylindrical skirt 322.

After the crimping clip 320 has been crimped, the flange and connection area 304, 306 is tightly positioned between crimping clip and septum with a force and/or form fit and is thus non-detachably connected to the dispense interface 200.

The needle guide 300 in FIG. 15 is also made of metal. Production of such a needle guide 300 can be facilitated particularly by producing the needle guide 300 by a deep drawing process.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(02)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(02)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(02)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (−150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab')2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

1-13. (canceled)

14. A needle guide configured to receive a needle in a first opening in an axial direction of the needle guide, comprising
a guide area comprising the first opening and a second opening,
a flange area and
a connection area,
wherein said connection area is configured to non-detachably connect said needle guide to a medical device and
wherein said needle guide is made of metal.

15. A needle guide according to claim 14, wherein at least said guide area is produced by deep drawing a metal sheet.

16. A needle guide according to claim 14, wherein said guide area is substantially cylindrical in the axial direction and tapers at least in parts from the first opening of guide area to the second opening of the guide area.

17. A needle guide according to claim 14, wherein the flange area is adjacent to the guide area, said flange area is substantially circular and said flange area is extending substantially perpendicular to the axial direction.

18. A needle guide according to claim 14, wherein said connection area extends substantially into the axial direction or perpendicular to the axial direction.

19. A needle guide according to claim 14, wherein said connection area comprises a plurality of projections separated by recesses and substantially projecting in the axial direction.

20. A needle guide according to claim 19, wherein said projections are angled compared to the axial direction.

21. A needle guide according to claim 19, wherein said projections are angled compared to the axial direction alternating towards the guide area and away from the guide area.

22. An apparatus comprising a needle guide according to claim 14, wherein said apparatus is a medical device configured to eject at least one medicament, or a dispense interface attachable to a medical device configured to eject at least one medicament.

23. A method to produce a needle guide according to claim 14, wherein said needle guide comprises a guide area, a flange area and a connection area, comprising the steps of deep drawing a metal sheet to produce the guide area.

24. A method according to claim 23, wherein said guide area, said flange area and said connection area are produced at least in part by said step of deep drawing said metal sheet.

25. A method according to claim 23, further comprising the step of pressing said needle guide into a medical device.

26. A method according to claim 25, wherein said medical device is a dispense interface, in particular a needle hub.
